(19) United States
(12) Patent Application Publication
(10) Pub. No.: US 2026/0212991 A1
(43) Pub. Date: Jul. 23, 2026

(54) JUST-IN-TIME MOBILE APPLICATION FOR THE PROMOTION OF HEALTH, WELLNESS, HEALING AND EMOTIONAL SUPPORT

(71) Applicant: Matthew Vernon Hanson, Sun Praire, WI (US)

(72) Inventor: Matthew Vernon Hanson, Sun Praire, WI (US)

(21) Appl. No.: 19/445,767

(22) Filed: Jan. 12, 2026

Related U.S. Application Data
(60) Provisional application No. 63/747,897, filed on Jan. 22, 2025.

(51) Int. Cl. G16H 20/70; G16H 20/30; H04L 51/046

(52) U.S. Cl. CPC G16H 20/70; G16H 20/30; H04L 51/046

(57) ABSTRACT

The invention disclosed herein is a mobile application and attendant website designed to promote health, wellness, healing, and emotional support. The platform leverages the unconditional love and support of close relationships to help users achieve various personal goals, such as weight loss, recovery from physical injury, or collective goals within a community. The application allows users to select specific goals and designate friends and relatives as their support network. These supporters provide encouragement through simple, positive feedback mechanisms, such as emojis and short text messages. The system is designed to be user-friendly and efficient, ensuring quick and meaningful interactions that foster a supportive environment. By focusing on the power of love and close relationships, this invention aims to provide a reliable support system for individuals who may not receive adequate support from society or professional networks.

### BACKGROUND OF THE INVENTION

Mankind is a web of needs (FIG. 1) and each person is a web of relationships (FIG. 2). Fulfilling needs and managing relationships successfully requires communication and a lot of ongoing support from others.

Body types, shapes, size, and physical deformities, injuries, disabilities and so on which results in bodies or mannerisms that “differ from the norm” can cause an individual an inordinate amount of stress, sadness, and have an outsized economic impact on their future.

That stress, sadness, and economic impact can then, in turn, result in less-than desirable life outcomes.

In other words, “success” in life is often highly dependent on others' perceptions of us.

Meaning that the “quality and quantity” of the things we get in life—whether in a career, in the number and depth of the relationships with significant others, or in any other endeavor—is highly dependent on how other people “see” us.

As a result “support,” doesn't always come from society or from co-workers.

In fact, most of the time, the opposite is true. Society, as a whole, is not a dependable support mechanism for the individual, either emotionally or economically.

In other words, if we are “different” or “outside the norm” and are shunned or set aside for various reasons, where does our support actually come from?

Primarily that support comes from those who love us without condition and can see past our physical ailments, body shape and size, and so on.

The invention herein disclosed is a platform for leveraging the love of those around us to support us in our goals—whether we are trying to lose weight, recover from physical injury, or reach a goal within a community. By leveraging the love of those around us—those who truly love us—it can help us to succeed in just about any endeavor.

### BRIEF SUMMARY OF THE INVENTION

The invention consists of a mobile application and attendant website which has several interlocking modules which allow the user to select a variety of goals, and then to select friends and relatives who will be their “cheering section” for those goals.

### BRIEF DESCRIPTION OF THE DRAWINGS

FIG. 1. Is a map of human needs

FIG. 2. Is a map of human relationships

FIG. 3. Is a line drawing of two gnomes, one fat, one thin

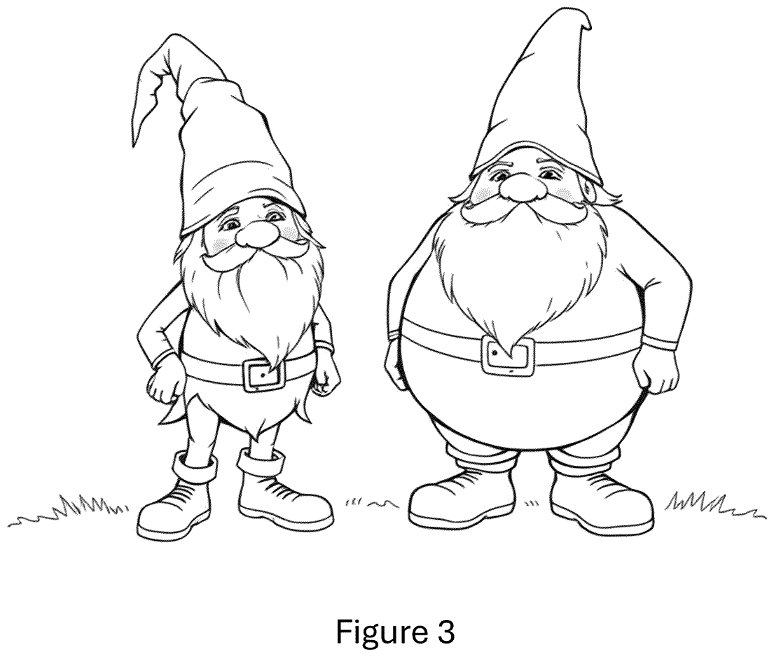

FIG. 4. Is an overview of the system

FIG. 5. Is a diagram of the user and supporter interactions

FIG. 6. Is a diagram of the user and supporter software functions

### DETAILED DESCRIPTION OF THE INVENTION

### The Relationship Problem

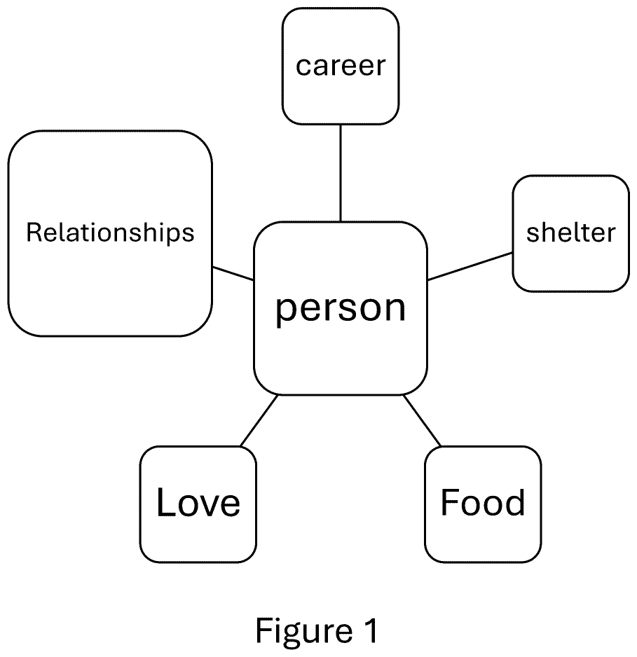

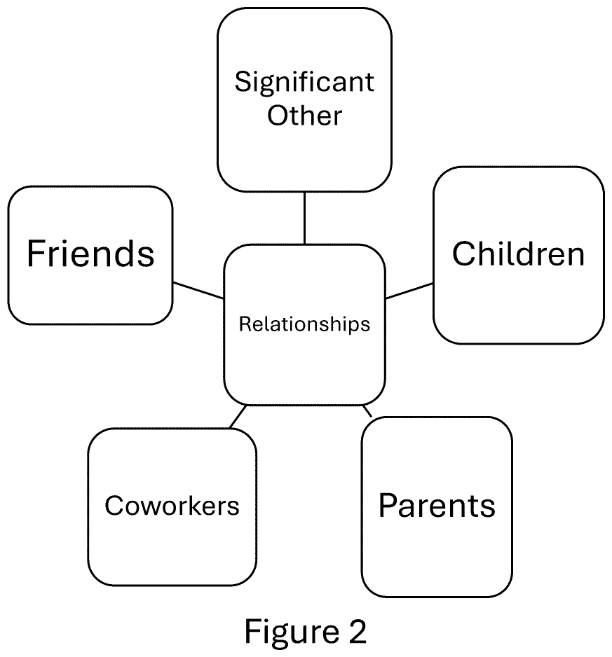

Mankind is a web of needs (FIG. 1) and each person is a web of relationships (FIG. 2). Creating fulfilling relationships and reaching obtainable goals in our lives requires a lot of communication and ongoing support from other people.

Body types, shapes, size, and physical deformities, injuries, disabilities and other physical or mental impairments which cause an individual to “differ from the norm” or to differ from what society considers “acceptable” can cause an individual an inordinate amount of stress, sadness, and have quantifiable economic impacts on their life over the long-term.

This stress, sadness, and cascade of economic impacts can, in turn, result in less-than desirable life outcomes. Including multiple goal failure, health and mental decline, substance abuse, and even suicide and premature death.

“Success” in life, while arguably desirable by all, is, for better or worse, highly dependent on others' perceptions of us. In many ways—“as we are seen, so we are.”

In other words, it doesn't matter how smart or capable we are—if no one will hire us.

It doesn't matter how willing we are to work if we can't obtain stable housing—because of how we look, talk, dress, or communicate.

It doesn't matter how kind or gentle we would be with a significant other if we are fat, or have a physical deformity, or a speech impediment, or are simply “outside the norm” of what a significant other deems desirable, expects, or is willing to accept for the long-term.

What this means is that the “quality and quantity” of the things we get in life—whether in a career, with significant others, or in any business endeavor—actually has a lot to do with how we look—and how others perceive (and judge) how we look.

Support from others—whether in attaining personal goals or in the normal course of business activity—usually doesn't come from society.

Nor does it typically come on any consistent basis from co-workers.

Or even from professional or community groups to which we belong.

Absence of support can be particularly glaring if we wrestle with weight issues, or a physical deformity, or disfiguring injury.

These issues cause an individual to vastly “differ from the norm”—causing a cascade of long-term problems.

When an individual “differs from the norm” an observer often makes a judgment about character based purely on physical appearance or manner of speaking, dress, or communication. And that observer's reaction can range from acceptance, to conditional acceptance, to instant and irrecoverable rejection.

This is not anyone's fault. Per a recent article discussing ophthalmology and perception “we are our eyes”—meaning that Mankind has visual systems that are highly integrated into the overall function of the brain.

As a result, our reaction to visual stimuli is immediate and, often, overwhelming. And that reaction is also emotional—and our initial impressions and feelings about something we see often stay with us for long periods of time.

This perceptive skill has a positive use when it comes to survival—identifying danger quickly and triggering a “fight or flight” response.

But the same type of “snap judgments” occur all the time across a wide variety of human endeavors.

Endeavors, in many cases, the success of which have everything to do with a person's skills, tenacity, and courage. And absolutely nothing to do with how they look.

But our perceptions of others—hijack how we feel about others. Even when visual appearance or manner makes absolutely no difference to the outcome of obtaining a particular goal.

And we have all, at times, found ourselves making snap judgments about others based on arbitrary physical criteria. And we often realize much later that those physical criteria have absolutely nothing to do with who a person actually is.

And the result of all of this is—people, good people, often find themselves completely bereft of support. In times of trouble. In times of disfiguring injury. In times when they are unemployed or otherwise unable to fend for themselves. Or when they wrestle with actual mental or physical disabilities.

And these people sometimes make a slow decline through the economic ranks, sometimes based on their appearance alone—fighting desperately to turn things around—but failing at every turn.

And there is no support for them at all. None. Not even a “thumbs up” or “we love you man” or “keep going, we've got your back.”

And for those of us who have had hard moments in our lives, or have suffered from horrific injury, or terrible illness, or have put on some weight, or been unemployed for long periods of time, little, tiny bits of encouragement, at just the right time, can mean the world.

But if we look a certain way, or talk a certain way, or act a certain way—and society isn't going to support us—who will?

In other words, if society isn't capable of providing support to an individual “who is outside the norm”—who can provide that support? Where does it come from?

The primary source of this support (and sometimes the only source of support) is from those who love us without condition or reservation. The people who and can see past our physical ailments, body shape and size, or disability—and love us anyway.

The people who love us without condition are not always “family” members—and everyone has a family member or two who is always “less than supportive” of our endeavors, or body shape, or life choices. That's just how human beings are.

But generally, most people have three or four people in the world who genuinely love them—without condition or equivocation. Friends. Relatives. Significant others. Children. And sometimes a medical or mental health professional or social worker who is a strong advocate when no one else can step in.

This invention seeks to leverage those three or four relationships to support an individual in the pursuit of a variety of life goals.

The initial purpose of the invention was to help an individual who was struggling to lose weight, and his friend who was homeless after a divorce and kept getting shuffled from place to place in a two-year odyssey as a nomad.

However, the platform does have a broader variety of uses which this patent seeks to cover.

Those additional uses include support when recovering from illness, recovering from serious injury (such as a soldier might experience after combat), or support in the pursuit of collective goals such as sports teams or and like-minded groups.

For example, if a running group is training for a marathon or a rowing club is preparing for a race, the app can create temporary groupings where people seek to help each other reach a specific goal over a relatively short time-period.

This differs from traditional social media platforms in a few key aspects in that this platform doesn't seek wide adoption and/or commentary by hundreds or thousands of people on an individual's activities.

The app actually limits support to those the user selects and KNOWS are\actually going to be supportive.

And then the platform seeks to leverage that support and keep that support positive by curtailing somewhat the depth and breadth of the responses available.

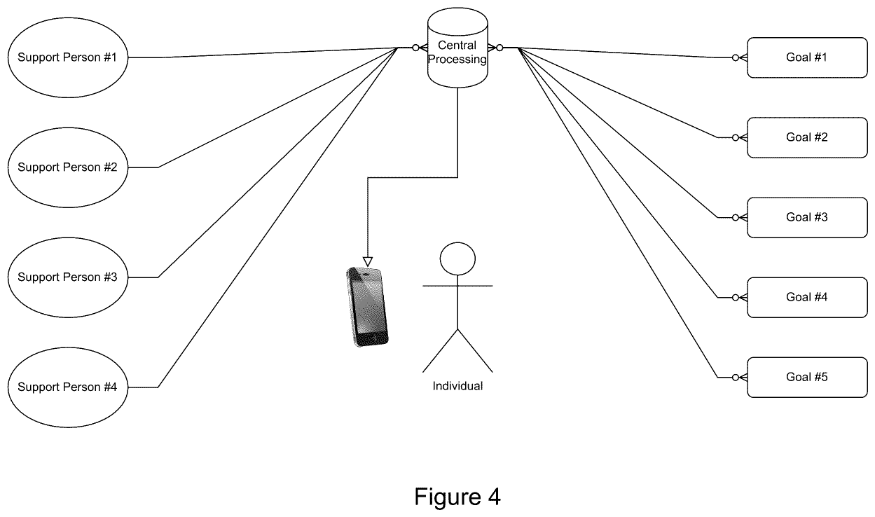

FIG. 4 shows an overall view of the system—where Support Persons #1- #5 are connected to a Central Processing Unit, which in turn is connected to User Goals #1- #5.

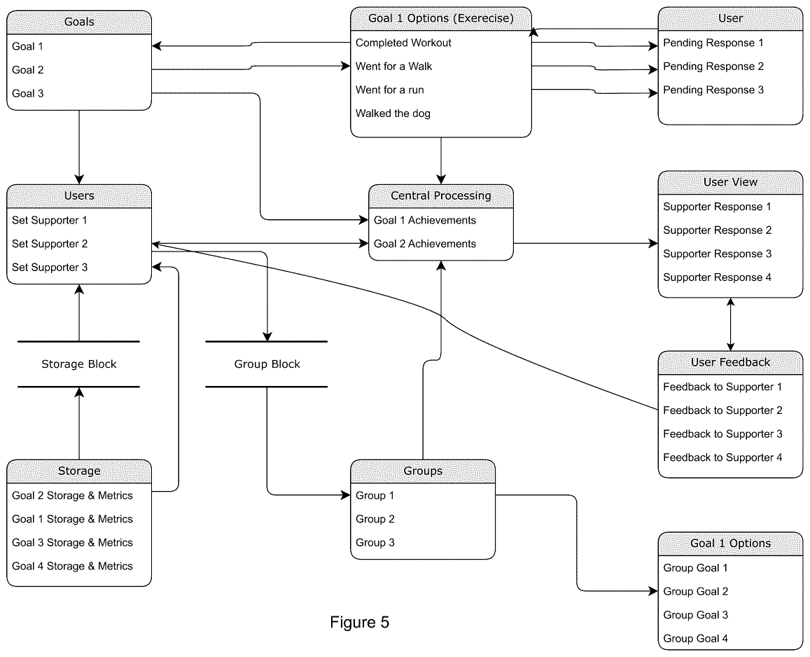

In FIG. 5, the User can assign support people to various goals. Meaning that when the User assigns a Support Person to a particular goal, at any point in the future when the User completes a task under that goal, that Support Person will receive an in-app message which will in turn appear on their phone's home screen.

When the User receives an in-app message, the responses are relatively simple in nature—thumbs up, heart, prayer, or other relevant emojis (which can be selected by the user) as well as a space for a short text message.

That emoji and/or text response is then returned to the user.

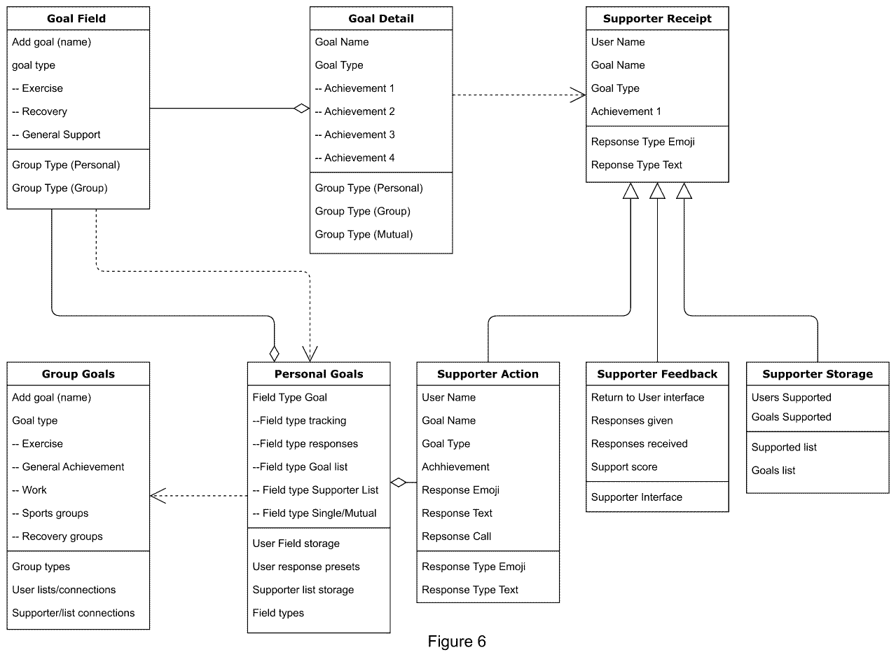

Under each Goal in FIG. 6, the user can name the goal, select specific emojis for support (or go with a standard set of emojis), and name specific actions related to the goal.

There is also the possibility here of having pre-populated lists for specific weight-loss, mental health, or physical recovery (from injury) specific tasks.

But in practice, it is probably a short two-or three item list like “completed workout” or “went for a walk” or “completed physical therapy” or “walked the dog.”

In keeping the interface simple for both the User and the Supporter—it drives very simple messaging which has a very low time-cost for both parties.

The ideal time from notification to response from the Supporter is under 5 seconds. And the ideal time from completion of a task to letting the Supporter's network know that a task has been completed—is also five seconds.

In shortening the amount of time required by both parties, it increases fluidity of response as well as overall adoption of the platform.

Texting capability is there but primarily would be used by the Supporter for short notes of encouragement.

The goal of the User—is to complete the task.

The goal of the Supporter is to provide support.

And both playing their part as simply and effectively as possible.

The invention herein disclosed is a platform for leveraging the love of those around us to support us in our goals—whether we are trying to lose weight, obtain a goal, or recover from physical injury. It also includes the possibility of supporting short-term goals through short-term formation of sports teams or other types of organizations attempting to achieve collective goals. By leveraging the love of those around us, it can help us to succeed.

### Underlying Philosophy

This invention leverages the power of love and close relationships to support health and fitness goals. Traditional perceptions of success often focus on physical appearance, but real success requires seeing beyond the surface. Only those with unconditional love—parents, spouses, children, or close friends—can offer the emotional support needed for long-term health and recovery and long-term meaningful achievement of health, fitness, and goals related to physical injury.

Health journeys are never solo. Whether through a gym, Peloton, or running club, support is crucial. However, no existing system fully utilizes love as the driving force for change. This platform focuses on creating a supportive network of people who genuinely care, excluding those who might judge based on appearance or lack emotional connection.

Relationships are central to health. Stress in one area impacts the whole mental and physical system of an individual. Meaningful communication, achievable goals, and mutual support are essential to changing that paradigm. Love provides the foundation for acceptance and encouragement at every stage of the journey in this invention.

The app enables users to share progress, like completing workouts, with their support network. Simple responses—claps, hearts, or short messages like “keep going!”—can make a big difference. Notifications prompt engagement, ensuring no one feels isolated. A single supportive message can be transformative, and can also be reversed as “supporter becomes supported.”

Mutual support is a key aspect of the invention. As users encourage others in their network, they receive support in return. Gentle reminders like “haven't seen you walking lately, you okay?” ensure no one falls through the cracks. Small gestures, like a parent's “I'm proud of you,” can inspire lasting motivation.

The platform is scalable, user-friendly, and integrates tools like Google Maps for tracking progress. It emphasizes positive feedback, with features like muting without unfriending. By fostering two-way engagement powered by love, this system ensures everyone is supported. Love drives both User and Supporter action, creating a healthier, more connected community where no one is left behind.

1: A mobile application and attendant website designed to promote health, wellness, healing, and emotional support, comprising:
a user interface that allows users to select specific personal goals;
a mechanism for designating friends and relatives as supporters;
a feedback system enabling supporters to provide encouragement through emojis and short text messages;
a central processing unit that connects users' goals with their designated supporters;
a notification system that alerts supporters when a user completes a task related to their goal, prompting a response from the supporter.

2: The mobile application and attendant website of claim 1, wherein the feedback system is configured to:
limit the depth and breadth of responses available to supporters to ensure positive and meaningful interactions;
include pre-populated lists for specific tasks related to weight loss, mental health, or physical recovery;
allow users to customize the emojis and short text messages used for feedback.

3: The mobile application and attendant website of claim 1, further comprising:
a feature that enables the formation of temporary groups for collective goals, such as sports teams or community projects;
a tracking system that integrates tools like Google Maps to monitor progress;
a muting feature that allows users to mute supporters without unfriending them, ensuring a supportive and non-judgmental environment.
